(19) United States
(12) Patent Application Publication
(10) Pub. No.: US 2005/0002892 A1
(43) Pub. Date: Jan. 6, 2005

(54) Conditioning composition comprising cationic crosslinked thickening polymer and nonionic surfactant

(75) Inventors: Golam Faruque Khan, Higashinada-ku (JP); Susan Marie Guskey, Higashinada-ku (JP); Rober Lee Wells, Higashinada-ku (JP)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/861,731

(22) Filed: Jun. 4, 2004

Related U.S. Application Data
(60) Provisional application No. 60/476,709, filed on Jun. 6, 2003.

(51) Int. Cl. A61K 7/06; A61K 7/11; A61K 7/075; A61K 7/08

(52) U.S. Cl. 424/70.17

(57) ABSTRACT

Disclosed is a conditioning composition comprising by weight: (a) from about 0.01% to about 5% of a cationic crosslinked thickening polymer; (b) from about 0.1% to about 10% of a surfactant selected from the group consisting of a nonionic surfactant, a cationic surfactant, and mixtures thereof; and (c) an aqueous carrier; and wherein the composition is substantially free of a water-insoluble high melting point oily compound. The conditioning composition of the present invention has a suitable rheology for conditioning compositions, and provides conditioning benefits. The composition is especially suitable for hair care products such as hair conditioning products for rinse-off use.

### CROSS REFERENCE TO RELATED APPLICATION

This application claims the benefit of U.S. Provisional Application No. 60/476,709, filed on Jun. 6, 2003.

### FIELD OF THE INVENTION

The present invention relates to a conditioning composition comprising a cationic crosslinked thickening polymer, a nonionic surfactant, and the composition being substantially free of water-insoluble high melting point oily compounds. The conditioning composition of the present invention has a suitable rheology for conditioning compositions and provides conditioning benefits. The composition is especially suitable for hair care products such as hair conditioning products for rinse-off use.

### BACKGROUND OF THE INVENTION

A variety of conditioning compositions such as hair conditioning compositions, skin conditioning compositions, and fabric softeners have been used for a variety of substances such as hair, skin, and fabric. A common method of providing conditioning benefits is through the use of conditioning agents such as cationic surfactants and polymers, high melting point fatty compounds, low melting point oils, silicone compounds, and mixtures thereof. Most of these conditioning agents are known to provide various conditioning benefits. For example, some cationic surfactants, when used together with some high melting point fatty compounds, are believed to provide a gel matrix which has a suitable rheology for conditioning compositions and which is suitable for providing a variety of conditioning benefits, especially when used for hair care products, such as slippery feel, softness and reduced tangling on wet hair and softness and moisturized feel on the hair when they are dried.

There exists a need for achieving the suitable rheology for conditioning compositions by other methods than forming the above gel matrix, while maintaining the conditioning benefits of the gel matrix.

Additionally, most of the above conditioning agents are also known to make the composition opaque. Thus, there is a need for conditioning compositions having a clear product appearance i.e., transparent or translucent product appearance.

Furthermore, most of the above conditioning agents are also known to weigh down the hair, when these conditioning agents are included in hair care compositions. For consumers who desire maintaining or increasing hair volume such as consumers having fine hair, the hair weighing down is not desirable. Thus, there is a need for hair conditioning compositions which do not weigh down the hair while providing conditioning benefits.

There also exists a need for conditioning compositions which consumers feel are easy to rinse-off while providing conditioning benefits, when the compositions are used in a form of rinse-off products. Conditioner compositions containing the above gel matrix also provide long-lasting slippery feel when rinsing the substance. Thus, there is a need for conditioning compositions which can easily leave the substance with a clean feel when rinsing the substance, while depositing sufficient amount of conditioning agents on the substance.

Based on the foregoing, there remains a need for conditioning compositions which have a suitable rheology for conditioning compositions by other methods than a gel matrix comprised by cationic surfactants and high melting point fatty compounds, while providing conditioning benefits, especially softness and reduced tangling on wet hair when used for hair care products such as hair conditioning products. There is also a need for such conditioning compositions which are suitable for providing further benefits such as, clear product appearance, not weighing down the hair, and easy to rinse-off feel, while providing the above rheological and conditioning benefits.

None of the existing art provides all of the advantages and benefits of the present invention.

### SUMMARY OF THE INVENTION

The present invention is directed to conditioning composition comprising by weight:
(a) from about 0.01% to about 5% of a cationic crosslinked thickening polymer,
(b) from about 0.1% to about 10% of a surfactant selected from the group consisting of a nonionic surfactant, a cationic surfactant, and mixtures thereof; and
(c) an aqueous carrier;
   and wherein the composition is substantially free of a water-insoluble high melting point oily compound.

These and other features, aspects, and advantages of the present invention will become better understood from a reading of the following description, and appended claims.

### DETAILED DESCRIPTION OF THE INVENTION

While the specification concludes with claims particularly pointing out and distinctly claiming the invention, it is believed that the present invention will be better understood from the following description.

Herein, “comprising” means that other steps and other ingredients which do not affect the end result can be added. This term encompasses the terms “consisting of” and “consisting essentially of”.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

Herein, “mixtures” is meant to include a simple combination of materials and any compounds that may result from their combination.

Compositions

The conditioning composition of the present invention comprises by weight:
(a) from about 0.01% to about 5% of a cationic crosslinked thickening polymer;
(b) from about 0.1% to about 10% of a surfactant selected from the group consisting of a nonionic surfactant, a cationic surfactant, and mixtures thereof, and
(c) an aqueous carrier;
   and wherein the composition is substantially free of a water-insoluble high melting point oily compound.

The conditioning compositions of the present invention have a suitable rheology for conditioning compositions and provide conditioning benefits, especially softness and reduced tangling on wet hair when used for hair care products such as hair conditioning products. The conditioning compositions of the present invention are suitable for providing further benefits such as clear product appearance, not weighing down the hair, and easy to rinse-off feel, while providing the above rheological and conditioning benefits. Thus, the composition of the present invention can provide clear product appearance in addition to the above theological and conditioning benefits. When used for hair care products, the composition of the present invention provides the above rheological and conditioning benefits while not weighing down the hair. Furthermore, when used in a form of rinse-off products, the composition of the present invention can provide easy to rinse-off feel while providing the above rheological and conditioning benefits.

It is believed that; the cationic crosslinked thickening polymer can provide a suitable rheology for conditioning compositions without the existence of a gel matrix comprised by cationic surfactants and high melting point fatty compound, while providing conditioning benefits, especially softness and reduced tangling on wet hair when used for hair care products such as hair conditioning products. The cationic crosslinked polymer is used in combination with surfactants, preferably nonionic surfactants in view of compatibility. By the use of nonionic surfactants, the composition can include additional ingredients such as silicone compounds, while not deteriorating the above benefits.

In the present invention, the composition being “substantially free of water-insoluble high melting point oily compound” means that the composition includes 1.0% or less, preferably 0.5% or less, more preferably 0.1% or less, still more preferably 0% of water-insoluble high melting point oily compounds. The water-insoluble high melting point oily compounds herein are those having a melting point of at least about 25° C., and a solubility in water at 25° C. of less than about 1 g/100 g water, preferably less than about 0.5 g/100 g water, more preferably less than about 0 g/100 g water. Such water-insoluble high melting point oily compound include, for example, fatty alcohols such as cetyl alcohol and stearyl alcohol, fatty acids such as stearic acid, fatty alcohol derivatives and fatty acid derivatives such as cetyl palmitate, hydrocarbons such as waxes, steroids such as cholesterol, and mixtures thereof.

Preferably, the composition of the present invention is transparent or translucent, and more preferably transparent. In the present invention, the composition being “transparent” means that the composition has a turbidity of no more than about 300 NTU (Nephelometric Turbidity Units), preferably no more than about 200 NTU, more preferably no more than about 100 NTU. In the present invention, the composition being “translucent” means that the composition has a turbidity of from about 300 NTU to about 3,000 NTU (Nephelometric Turbidity Units). The NTU values are measured using the Hach 2100N Laboratory Turbidimeter calibrated with Formazin standards, available from Hach Company.

Preferably, the composition of the present invention is substantially free of components which are incompatible to the cationic crosslinked polymer. It is believed that; such incompatible components undesirably interact with the cationic crosslinked polymer, and may deteriorate the rheological benefit of the cationic crosslinked polymer. Such components include, for example, anionic surfactants. Some levels and/or types of water-soluble cationic surfactants and water-soluble chelating agents may also undesirably interact with the cationic crosslinked polymer. In the present invention, the composition being “substantially free of incompatible components” means that the composition includes 1% or less, preferably 0.5% or less, more preferably 0% of such components.

Cationic Crosslinked Thickening Polymer

The compositions of the present invention comprise a cationic crosslinked thickening polymer. The cationic crosslinked thickening polymers are cationic polymers crosslinked by a crosslinking agent.

The cationic crosslinked thickening polymers useful herein those which can be dissolved in aqueous carrier and can provide appropriate viscosity and rheology properties to the composition, so that the composition of the present composition has a suitable viscosity, preferably from about 100 cps to about 100,000 cps, more preferably from about 1,000 cps to about 50,000 cps, still more preferably from about 2,000 cps to about 50,000 cps, even more preferably from about 5,000 cps to about 20,000 cps. The viscosity herein can be suitably measured by Brookfield RVT at a shear rate of 2·s⁻¹ at 26.7° C.

The cationic crosslinked thickening polymer is included in the composition of the present invention at a level by weight of from about 0.01% to about 5%, preferably from about 0.01% to about 3%, more preferably from about 0.05% to about 2%, still more preferably from about 0.1% to about 1%.

Preferably, the cationic crosslinked polymers useful herein have a crosslinked level of from about 5 ppm to about 5,000 ppm. In the present invention, “crosslinked level” means the amount of crosslinking agents of the total weight of the polymer on a weight/weight basis. Widely varying amounts of the crosslinking agents can be employed depending upon the properties desired in the final polymer, e.g. viscosifying effect. The crosslinking agents comprise, preferably from about 5 ppm to about 5,000 ppm, more preferably from about 50 ppm to about 1,000 ppm, still preferably from about 55 ppm to about 600 ppm, even more preferably from about 60 ppm to about 250 ppm of the total weight of the polymer on a weight/weight basis, in view of providing conditioning benefits such as slippery feel on wet substances.

Cationic crosslinked polymers useful herein include, for example, those comprising the monomer units and has the formula (A)ₘ(B)ₙ(C)ₚ wherein: (A) is a quaternized dialkylamninoalkyl acrylate, an acid addition salt of a dialkylaminoalkyl acrylate, or mixtures thereof; (B) is a quaternized dialkylaminoalkyl methacrylate, an acid addition salt of a dialkylaminoalkyl methacrylate, or mixtures thereof; (C) is a nonionic monomer polymerizable with (A) or (B); m, n, and p are independently zero or greater, but at least one of m or n is one or greater.

The monomer (C) can be selected from any of the commonly used monomers. Non-limiting examples of these monomers include acrylamide; methacrylamide; acrylate esters and methacrylate esters such as methylmethacrylate, 2-ethylhexylmethacrylate, and t-butylacrylate; (meth)acrylamide derivatives such as N-isopropylacrylamide, N, N-dimethyl acrylamide; and styrene. In the present invention, the monomer (C) is preferably acrylamide.

The alkyl portions of the monomers (A) and (B) are preferably short chain length alkyls such as C₁-C₈, more preferably C₁-C₅, still more preferably C₁-C₃, even still more preferably C₁-C₂. When quaternized, the polymers are preferably quaternized with short chain alkyls, i.e., C₁-C₈, more preferably C₁-C₅, still more preferably C₁-C₃, even still more preferably C₁-C₂. The acid addition salts refer to polymers having protonated amino groups. Acid addition salts can be performed through the use of halogen (e.g. chloride), acetic, phosphoric, nitric, citric, or other acids. In the present invention, (A) is preferably a chloride salt of trialkylaminoethyl acrylate, and more preferably a chloride salt of trimethylaminoethyl acrylate. In the present invention, (B) is preferably a chloride salt of trialkylaminoethyl methacrylate, and more preferably a chloride salt of trimethylaminoethyl methacrylate.

When the polymer contains the monomer (C), the molar proportion of the monomer (C) can be up to about 80% based on the total molar proportions of the monomers (A), (B), and (C). The molar proportions of (A) and (B) can independently be from 0% to about 100%. When acrylamide is used as the monomer (C), it will preferably be included at a level of from about 5% to about 80%.

The crosslinked polymers also contain a crosslinking agent, which is typically a material containing two or more unsaturated functional groups. The crosslinking agent is reacted with the monomer units of the polymer and is incorporated into the polymer, forming either links or covalent bonds between two or more individual polymer chains or between two or more sections of the same polymer chain. Nonlimiting examples of suitable crosslinking agents include those selected from the group consisting of methylenebisacrylamides, diacrylates, dimethacrylates, di-vinyl aryl (e.g. di-vinyl phenyl ring) compounds, polyalkenyl polyethers of polyhydric alcohols, and allyl acrylates. Specific examples of crosslinking agents useful herein include those selected from the group consisting of methylenebisacrylamide, ethylene glycol di-(meth)acrylate, propylene glycol di-(meth)acrylate, butylene glycol di-(meth)acrylate, 1,4-di-ethylene benzene, and allyl acrylate. Preferred herein is methylenebisacrylamide.

Exemplary, the crosslinked polymers useful herein include those conforming to the general structure (A)ₘ(B)ₙ(C)ₚ wherein m is zero, (B) is methyl quaternized dimethylaminoethyl methacrylate, (C) is acrylamide, and the crosslinking agent is methylenebisacrylamide. An example of such a crosslinking polymer is one that has the CTFA designation, “Polyquaternium 32”. Commercially available polyquaternium-32 useful herein include, for example, the one available as a mineral oil dispersion under the tradename Salcare® SC92 from Allied Colloids Ltd.

More preferred cationic crosslinked polymers useful herein include those not containing acrylamide or other monomer (C), i.e. p is zero. In these polymers, the monomers (A) and (B) are as described above. An especially preferred group of these polymers is one in which m is also zero. In this instance, the polymer is essentially a homopolymer of dialkylaminoalkyl methacrylate monomer or its quaternary ammonium or acid addition salt. These dialkylaminoalkyl methacrylate copolymers and homopolymers also contain a crosslinking agent as described above.

Highly preferred cationic crosslinked polymer is a homopolymer which does not contain acrylamide or other monomer (C). The homopolymers useful herein can be those conforming to the general structure (A)ₘ(B)ₙ(C)ₚ wherein m is zero, (B) is methyl quaternized dimethylamninoethyl methacrylate, p is zero, and the crosslinking agent is methylenebisacrylamide. An example of such a homopolymer is one that has the CTFA designation, “Polyquaternium 37”. Commercially available such polyquaternium-37 useful herein include, for example, the one available as a neat material, under the tradenames Synthalen CR, Synthalen CU, and Synthalen CN, all from 3V Sigma. Commerically available polyquaternium-37 useful herein also include, for example, the one available as a mineral oil dispersion, under the tradename Salcare® SC95, and the one available as an ester dispersion, wherein the ester can be propylene glycol dicaprylate/dicaprate and the dispersion can include various dispersing aids such as PPG-1 trigriceth-6, under the tradename Salcare® SC96, all from Allied Colloids Ltd. The polyquaternium-37 under the tradename Synthalen series have a crosslinked level of 50 ppm or more, while those under the tradename Salcare series have a crosslinked level of lower than 50 ppm.

Surfactant

The compositions of the present invention comprises a surfactant selected from the group consisting of a nonionic surfactant, a cationic surfactant, and mixtures thereof. The surfactant is included in the composition at a level by weight of from about 0.1% to about 10%, preferably from about 0.5% to about 8%, more preferably from about 1% to about 5%.

Nonionic Surfactant

The surfactant used in the compositions of the present invention is preferably a nonionic surfactant. Preferably, in the composition of the present invention, the nonionic surfactant is water-soluble in view of providing clear product appearance. By “water soluble” surfactants, what is meant is a surfactant which is sufficiently soluble in water to form a substantially clear solution to the naked eye at a concentration of 0.05%, preferably 0.1%, more preferably 0.2% in water (distilled or equivalent) at 25° C. Such water-soluble surfactants have an HLB value of from about 9 to about 18, more preferably from about 11 to about 18, still more preferably from about 13 to about 15.

A variety of nonionic surfactants can be used in the composition of the present invention. Such nonionic surfactants include, for example, polyoxyethylene alkyl ethers; polysorbates such as polysorbate-20 through 80; polyethylene glycol derivatives of glycerides such as polyethylene glycol derivatives of hydrogenated castor oil including PEG40 through 100 hydrogenated castor oil, and polyethylene glycol derivatives of stearic acid including PEG-10 through 55 stearate.

Among a variety of nonionic surfactants, preferred are polyoxyethylene alkyl ethers. Especially preferred polyoxyethylene alkyl ether useful herein are those having an C12-C22 alkyl chain, and having the above HLB value. Highly preferred polyoxyethylene alkyl ethers include, for example, laureth series of compounds such as laureth-7 through 12; ceteth series of compounds such as ceteth-7 through 20; and pareth series of compounds such as pareth-9 through 15.

Cationic Surfactant

The compositions of the present invention may contain a cationic surfactant. The cationic surfactants useful herein include, for example, those corresponding to the general formula (I): 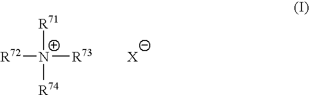
wherein at least one of R⁷¹, R⁷², R⁷³ and R⁷⁴ is selected from an aliphatic group of from 8 to 30 carbon atoms or an aromatic, alkoxy, polyoxyalkylene, alkylamido, hydroxyalkyl, aryl or alkylaryl group having up to about 22 carbon atoms, the remainder of R⁷¹, R⁷², R⁷³ and R⁷⁴ are independently selected from an aliphatic group of from 1 to about 22 carbon atoms or an aromatic, alkoxy, polyoxyalkylene, alkylamido, hydroxyalkyl, aryl or alkylaryl group having up to about 22 carbon atoms; and X is a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The aliphatic groups can contain, in addition to carbon and hydrogen atoms, ether linkages, and other groups such as amino groups. The longer chain aliphatic groups, e.g., those of about 12 carbons, or higher, can be saturated or unsaturated. Preferred is when R⁷¹, R⁷², R⁷³ and R⁷⁴ are independently selected from C₁ to about C₂₂ alkyl. Nonlimiting examples of cationic surfactants useful in the present invention include the materials having the following CTFA designations: quaternium-8, quaternium-14, quatemium-18, quaternium-18 methosulfate, quaternium-24, and mixtures thereof.

Among the cationic surfactants of general formula (I), preferred are those containing in the molecule at least one alkyl chain having at least 16 carbons. Nonlimiting examples of such preferred cationic surfactants include: behenyl trimethyl ammonium chloride available, for example, with tradename Genamine KDMP from Clariant, with tradename INCROQUAT TMC-80 from Croda and ECONOL TM22 from Sanyo Kasei; cetyl trimethyl ammonium chloride available, for example, with tradename CA-2350 from Nikko Chemicals, hydrogenated tallow alkyl trimethyl ammonium chloride, dialkyl (14-18) dimethyl ammonium chloride, ditallow alkyl dimethyl ammonium chloride, dihydrogenated tallow alkyl dimethyl ammonium chloride, distearyl dimethyl ammonium chloride, dicetyl dimethyl ammonium chloride, di(behenyl/arachidyl) dimethyl ammonium chloride, dibehenyl dimethyl ammonium chloride, stearyl dimethyl benzyl ammonium chloride, stearyl propyleneglycol phosphate dimethyl ammonium chloride, stearoyl amidopropyl dimethyl benzyl ammonium chloride, stearoyl amidopropyl dimethyl (myristylacetate) ammonium chloride, and N-(stearoyl colamino formyl methyl) pyridinium chloride.

Salts of tertiary fatty amines are also suitable for cationic surfactants. The alkyl groups of such amines preferably have from about 12 to about 22 carbon atoms, and can be substituted or unsubstituted. Particularly useful are amidoamines of the following general formula:
R¹CONH(CH₂)ₘN(R²)₂
wherein R¹ is a residue of C₁₁ to C₂₄ fatty acids, R² is a C₁ to C₄ alkyl, and m is an integer from 1 to 4.

Preferred amidoamine useful in the present invention includes stearamidopropyldimethylamine, stearamidopropyldiethylamine, stearamidoethyldiethylamine, stearamidoethyldimethylamine, palmitamidopropyldimethylamine, palmitamidopropyldiethylamine, palmitamidoethyldiethylamine, palmitamidoethyldimethylamine, behenamidopropyldimethylamine, behenamidopropyldiethylamine, behenamidoethyldiethylamine, behenamidoethyldimethylamine, and mixtures thereof; more preferably stearamidopropyldimethylamine, stearamidoethyldiethylamine, and mixtures thereof.

The amidoamines herein are preferably used in combination with acids selected from the group consisting of L-glutamic acid, lactic acid, hydrochloric acid, malic acid, succinic acid, acetic acid, fumaric acid, L-glutamic acid hydrochloride, tartaric acid, citric acid, and mixtures thereof; preferably L-glutamic acid, lactic acid, hydrochloric acid, and mixtures thereof. Preferably, the mole ratio of amidoamine to acid is from about 1:0.3 to about 1:2, more preferably from about 1:0.5 to about 1:1.3.

Among the above cationic surfactants, preferred are water-insoluble cationic surfactants in view of less incompatibility with the cationic crosslinked polymer. Such water-insoluble cationic surfactants useful herein include, for example, quaternized ammonium salts having at least two long alkyl chains such as dialkyl (14-18) dimethyl ammonium chloride, ditallow alkyl dimethyl ammonium chloride, dihydrogenated tallow alkyl dimethyl ammonium chloride, distearyl dimethyl ammonium chloride, dicetyl dimethyl ammonium chloride, di(behenyl/arachidyl) dimethyl ammonium chloride, and dibehenyl dimethyl ammonium chloride.

Aqueous Carrier

The compositions of the present invention comprise an aqueous carrier. The level and species of the carrier are selected according to the compatibility with other components, and other desired characteristic of the product.

Carriers useful in the present invention include water and water solutions of lower alkyl alcohols. Lower alkyl alcohols useful herein are monohydric alcohols having 1 to 6 carbons, more preferably ethanol and isopropanol.

Preferably, the aqueous carrier is substantially water. Deionized water is preferably used. Water from natural sources including mineral cations can also be used, depending on the desired characteristic of the product. Generally, the compositions of the present invention comprise from about 20% to about 99%, preferably from about 40% to about 98%, and more preferably from about 50% to about 98% water.

The pH of the present composition is preferably from about 3 to about 9, more preferably from about 3 to about 7. Buffers and other pH adjusting agents can be included to achieve the desirable pH.

Additional Thickening Polymer

The compositions of the present invention preferably contain an additional thickening polymer. The additional thickening polymers useful herein are those different from the polymers disclosed above under the title “CATIONIC CROSSLINKED THICKENING POLYMER”. The additional thickening polymers useful herein are those which can be dissolved in aqueous carrier and, together with the above cationic crosslinked thickening polymer, can provide appropriate viscosity and rheology properties to the composition, so that the composition of the present composition has a suitable viscosity.

The additional thickening polymer can be included in the composition of the present invention at a level by weight of, preferably from about 0.01% to about 5%, more preferably from about 0.05% to about 3%, still more preferably from about 0.1% to about 2%, still more preferably from about 0.3% to about 1.5%.

A variety of additional thickening polymers can be used in the compositions of the present invention. Thickening polymers useful herein include, for example, cellulose and its derivatives such as cellulose ethers, hydrophobically modified cellulose ethers, and quaternized celluloses; guar gums including cationic guar gums and nonionic guar gums; crosslinked polymers such as nonionic crosslinked polymers and cationic crosslinked polymers; and acrylate polymers such as sodium polyacrylate, polyethylacrylate, and polyacrylamide. The thickening polymers useful herein may include the polymers disclosed below under the title “CATIONIC CONDITIONING POLYMER”.

Among a variety of additional thickening polymers, nonionic polymers are preferably used in the compositions of the present invention, in view of providing clean usage feel. More preferably, the additional thickening polymer is selected from the group consisting of a nonionic guar gum, a nonionic cellulose, and mixtures thereof, in view of compatibility with the cationic crosslinked polymer.

The nonionic guar gum useful herein has a molecular weight of preferably from about 500,000 to about 4,000,000, more preferably from about 1,000,000 to about 3,000,000. Commercially available nonionic guar gums useful herein include, for example, that having a molecular weight of about 2,000,000 available from Rhodia with a tradename Jaguar HP-105.

The nonionic cellulose polymers useful herein include, for example, hydroxymethyl cellulose, hydroxyethyl cellulose, hydroxyethylethylcellulose, cetyl hydroxyethylcellulose. Among them, highly preferred is hydroxyethylethylcellulose. Commercially available nonionic celluloses useful herein include, for example, hydroxyethylethyl cellulose with a tradename Elfacos CD481 available from Akzo Nobel, cetyl hydroxyethyl cellulose with a tradename of Polysurf 67 available from Aqualon.

Silicone Compound

The compositions of the present invention preferably contain a silicone compound. The silicone compounds herein are preferably used at levels by weight of the compositions of from about 0.1% to about 10%, more preferably from about 0.1% to about 8%, still more preferably from about 0.5% to about 5%.

Among a variety of silicone compounds, preferred are water-soluble or a nanoemulsion. In the present invention, a “water-soluble” silicone compound means that the silicone compound has a solubility in water at 25° C. of at least 0.05 g/100 g water, preferably at least 0.1 g/100 g water, more preferably at least 0.2 g/100 g water. In the present invention, a “water-soluble” silicone compound also means that the silicone compound is solublized in water under the existence of surfactants. Dimethicone copolyols, amodimethicone copolyols, and quaternized silicones can be water-soluble depending on the level of alkoxylate chains and quatemized groups in their structure. Commercially available water-soluble silicone compounds useful herein include, for example, dimethicone copolyol with a tradename Silicone DC-5330, and amidomethicone copolyol with tradenames Silicone BY16-906 and aminomethicone copolyol DC2-8500, all available from Dow Corning. In the present invention, a “nanoemulsion” of silicone compound means that the silicone compound is dispersed in the composition in the form of emulsion having an average diameter of 200 nm or less. Commercially available silicone compounds in the form of a nanoemulsion useful herein includes, for example, that with a tradename Silicone DC-8177 available from Dow Coming; quatemized silicone nanoemulsion with a tradename DC5-7133 available from Dow Coming; and amodimethicone nanoemulsion with a tradename XS65-B6413 available from General Electric.

Highly preferred water-soluble silicone compounds useful herein are hydrophobically modified amidomethicone copolyol having a following formula: 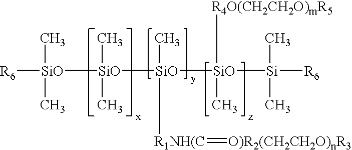
wherein R₁, R₂, R₄ are respectively C1-C3 alkyl, preferably ethyl; R₃ is an alkyl group having 8-22 carbon atoms, preferably 10-20 carbon atoms, more preferably 12-16 carbon atoms, even more preferably 12 carbon atoms; R₅ is H or C1-C3 alkyl, preferably methyl; R₆ is OH or CH₃, preferably methyl; n is an integer of 1-10, highly preferably 5; m is an integer of 2-20, highly preferably 12; n+m=3-30, preferably 5-25, more preferably 8-20, even more preferably 17; x is an integer from 200 to 500, preferably from 300 to 400; y is an integer from 5 to 40, preferably from 10 to 30; and z is 0 or an integer from 1 to 30, preferably from 5 to 20. Commercially available hydrophobically modified amidomethicone copolyols useful herein include, for example, that available from Dow Corning with a tradename BY 16-906.

Other silicones having conditioning properties may also be used in the composition. Such silicones include, for example, polyalkyl siloxanes such as polydimethylsiloxane from General Electric Company in their TSF 451 series and from Dow Corning in their Dow Corning SH200 series; polyaryl siloxanes; polyalkylaryl siloxanes; polyether siloxane copolymers; amino substituted silicones such as amodimethicone with tradename BY16-872 available from Dow Corning; quaternized silicones such as that available from Union Carbide under the tradename UCAR SILICONE ALE 56 and that available from Noveon with a tradename Ultrasil Q-Plus; and mixtures thereof. The silicone compounds for use herein will preferably have a viscosity of from about 100 to about 2,000,000 centistokes at 25° C. The viscosity can be measured by means of a glass capillary viscometer as set forth in Dow Corning Corporate Test Method CTM0004, Jul. 20, 1970.

Other Additional Ingredients

The composition of the present invention may include other additional components, which may be selected by the artisan according to the desired characteristics of the final product and which are suitable for rendering the composition more cosmetically or aesthetically acceptable or to provide them with additional usage benefits. Such other additional components generally are used individually at levels of from about 0.001% to about 10%, preferably up to about 5% by weight of the composition.

Cationic Conditioning Polymer

The conditioning compositions of the present invention may contain cationic conditioning polymers. The cationic polymers useful herein are those which can be dissolved in aqueous carrier and generally have a weight average molecular weight which is at least about 5,000, typically at least about 10,000, and is less than about 10 million, preferably, the molecular weight is from about 100,000 to about 2 million. The additional cationic conditioning polymers useful herein are those different from the polymers disclosed above under the title “CATIONIC CROSSLINKED THICKENING POLYMER”. The cationic polymers useful herein may include the polymers disclosed above under the title “ADDITIONAL THICKENING POLYMER”.

The cationic conditioning polymer can be included in the compositions at a level by weight of preferably from about 0.01% to about 10%, more preferably from about 0.05% to about 5%.

Suitable cationic conditioning polymers include, for example: copolymers of 1-vinyl-2-pyrrolidone and 1-vinyl-3-methylimidazolium salt (e.g., chloride salt) (referred to in the industry by the Cosmetic, Toiletry, and Fragrance Association, “CTFA”, as Polyquaternium-16), such as those commercially available from BASF Wyandotte Corp. (Parsippany, N.J., USA) under the LUVIQUAT tradename (e.g., LUVIQUAT FC 370); copolymers of 1-vinyl-2-pyrrolidone and dimethylaminoethyl methacrylate (referred to in the industry by CTFA as Polyquatemium-11) such as those commercially available from Gaf Corporation (Wayne, N.J., USA) under the GAFQUAT tradename (e.g., GAFQUAT 755N); cationic diallyl quaternary ammonium-containing polymers, including, for example, dimethyldiallylammonium chloride homopolymer and copolymers of acrylamide and dimethyldiallylammonium chloride, referred to in the industry (CTFA) as Polyquaternium 6 and Polyquaternium 7, Polyquaternium-7 including that commercially available with a tradename Merquat 550 from Ondeo Nalco; and polymethacrylamidopropyl trimonium chloride such as that commercially available with a tradename Polycare 133 from Rhone-Poulenc.

Also suitable cationic conditioning polymers herein include cationic cellulose derivatives. Cationic cellulose derivative useful herein include, for example, salts of hydroxyethyl cellulose reacted with trimethyl ammonium substituted epoxide, referred to in the industry (CTFA) as Polyquaternium 10, available from Amerchol Corp. (Edison, N.J., USA) in their Polymer JR® and LR® series, and also available from National Starch & Chemical with a tradename Celquat SC-230M; polymeric quaternary ammonium salts of hydroxyethyl cellulose reacted with lauryl dimethyl ammonium-substituted epoxide, referred to in the industry (CTFA) as Polyquaternium 24, available from Amerchol Corp. (Edison, N.J., USA) under the tradename Polymer LM-200®; and Polyquatemium-4 with tradename Celquat H-100 available from National Starch & Chemical.

Other suitable cationic conditioning polymers include cationic guar gum derivatives, such as guar hydroxypropyltrimonium chloride commercially available from Rhodia in their Jaguar R series.

Humectant

The compositions of the present invention may contain a humectant. The humectants herein are selected from the group consisting of polyhydric alcohols, water soluble alkoxylated nonionic polymers, and mixtures thereof. The humectants herein are preferably used at levels by weight of the composition of from about 0.1% to about 20%, more preferably from about 0.5% to about 5%.

Polyhydric alcohols useful herein include glycerin, sorbitol, propylene glycol, butylene glycol, hexylene glycol, ethoxylated glucose, 1, 2-hexane diol, hexanetriol, dipropylene glycol, erythritol, trehalose, diglycerin, xylitol, maltitol, maltose, glucose, fructose, sodium chondroitin sultate, sodium hyaluronate, sodium adenosin phosphate, sodium lactate, pyrrolidone carbonate, glucosamine, cyclodextrin, and mixtures thereof.

Water soluble alkoxylated nonionic polymers useful herein include polyethylene glycols and polypropylene glycols having a molecular weight of up to about 10,000 such as those with CTFA names PEG-4, PEG-8, PEG-12, PEG-20, PEG-150 and mixtures thereof.

Other Components

A wide variety of other additional components can be formulated into the present compositions. These include: other conditioning agents such as hydrolysed collagen with tradename Peptein 2000 available from Hormel, vitamin E with tradename Emix-d available from Eisai, panthenol available from Roche, panthenyl ethyl ether available from Roche, nonionic surfactants such as glyceryl stearate available from Stepan Chemicals, hydrolysed keratin, proteins, plant extracts, and nutrients; emollients such as PPG-3 myristyl ether with tradename Varonic APM available from Goldschmidt, Trimethyl pentanol hydroxyethyl ether, PPG-11 stearyl ether with tradename Varonic APS available from Goldschmidt, Stearyl heptanoate with tradename Tegosoft SH available from Goldschmidt, Lactil (mixture of Sodium lactate, Sodium PCA, Glycine, Fructose, Urea, Niacinamide, Inositol, Sodium Benzoate, and Lactic acid) available from Goldschmidt, Ethyl hexyl palmitate with tradename Saracos available from Nishin Seiyu and with tradename Tegosoft OP available from Goldschmidt; hair-fixative polymers such as amphoteric fixative polymers, cationic fixative polymers, anionic fixative polymers, nonionic fixative polymers, and silicone grafted copolymers; preservatives such as benzyl alcohol, methyl paraben, propyl paraben and imidazolidinyl urea; coloring agents, such as any of the FD&C or D&C dyes; hair oxidizing (bleaching) agents, such as hydrogen peroxide, perborate and persulfate salts; hair reducing agents such as the thioglycolates; perfumes; ultraviolet and infrared screening and absorbing agents such as octyl salicylate; antidandruff agents such as zinc pyrrithione and salicylic acid; visible particles with tradenames Unisphere and Unicerin available from Induchem AG (Switzerland); and anti-foaming agent such as that with a tradename XS63-B8929 available from GE-Toshiba Silicone.

Product Forms

The conditioning compositions of the present invention can be in the form of rinse-off products or leave-on products, can be transparent, translucent, or opaque, and can be formulated in a wide variety of product forms, including but not limited to creams, gels, emulsions, mousses and sprays.

The conditioning compositions of the present inventions can be used for conditioning a variety of substances such as hair, skin, and fabric, by applying the compositions to the substances such as hair, skin, and fabric. The conditioning composition of the present invention is especially suitable for hair care products such as hair conditioners, skin care products such as skin conditioners, and fabric care products such as fabric softeners.

The conditioning compositions of the present invention are especially suitable for hair conditioners for rinse-off use. Such compositions are preferably used by following steps:
(i) after shampooing hair, applying to the hair an effective amount of the conditioning composition for conditioning the hair; and
(ii) then rinsing the hair.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. Ingredients are identified by chemical or CTFA name, or otherwise defined below.

Compositions (wt %)

| | Ex. 1 | Ex. 2 | Ex. 3 | Ex. 4 | Ex. 5 | Ex. 6 | Ex. 7 |
|---|---|---|---|---|---|---|---|
| Cationic crosslinked | 0.3 | 0.3 | 0.3 | 0.5 | 0.7 | — | 0.4 |
| polymer-1*1 | | | | | | | |
| Cationic crosslinked | — | — | — | — | — | 0.3 | — |
| polymer-2*2 | | | | | | | |
| Laureth-9 | 3 | 2 | 2 | 2 | — | 3 | — |
| Pareth-12 | — | — | — | — | 2 | — | — |
| Ceteth-7 | — | — | — | — | — | — | 3 |
| Ceteth-15 | — | 1 | — | — | — | — | — |
| Ceteth-20 | — | — | 1 | — | — | — | — |
| Nonionic guar gum*3 | 0.75 | 0.75 | 0.75 | 0.4 | — | 0.75 | 0.5 |
| Amidomethicone | 3 | 3 | 3 | 4 | 4 | 3 | 2 |
| copolyol*4 | | | | | | | |
| Silicone | — | — | — | — | — | — | 1 |
| nanoemulsion*5 | | | | | | | |
| Antifoaming agent*6 | 0.075 | — | 0.075 | 0.075 | 0.075 | — | — |
| Vitamin E*7 | — | — | — | — | — | — | 0.05 |
| Hydrolyzed collagen*8 | — | — | — | — | — | — | 0.01 |
| Panthenyl ethyl | — | — | — | — | — | 0.05 | 0.05 |
| ether*9 | | | | | | | |
| Panthenol*10 | — | — | — | — | — | 0.05 | 0.05 |
| Benzophenone-4*11 | — | — | — | — | — | — | 0.1 |
| Octyl | — | — | — | — | — | — | 0.2 |
| Methoxycinnamate*12 | | | | | | | |
| Benzyl alcohol | — | 0.4 | 0.4 | — | 0.4 | 0.4 | 0.4 |
| Methyl- | 0.03 | 0.03 | — | 0.03 | 0.03 | — | — |
| chloroisothiazolinone/ | | | | | | | |
| Methyl- | | | | | | | |
| isothiazolinone*13 | | | | | | | |
| Methyl Paraben | 0.2 | — | 0.2 | 0.2 | 0.2 | — | 0.2 |
| Phenoxyethanol | — | — | 0.3 | — | — | — | 0.3 |
| Perfume solution | 0.25 | 0.25 | 0.25 | 0.30 | 0.30 | 0.35 | 0.50 |

| | |
|---|---|
| Deionized Water | q.s. to 100% |

Definitions of Components
*1Cationic crosslinked polymer-1: Polyquaternium-37 available from 3V Sigma with a tradename Synthalen CR
*2Cationic crosslinked polymer-2: Polyquaternium-37 available from 3V Sigma with a tradename Synthalen CU
*3Nonionic guar gum: Jaguar HP-105 having a molecular weight of about 2,000,000 available from Rhodia
*4Amidomethicone copolyol: Silicone BY16-906 available from Dow Corning
*5Silicone nanoemulsion: Silicone DC-8177 available from Dow Corning
*6Anti-foaming agent: XS63-B8929 available from GE-Toshiba Silicone
*7Vitamin E: Emix-d Available from Eisai
*8Hydrolyzed collagen: Peptein 2000 available from Hormel
*9Panthenyl ethyl ether available from Roche
*10Panthenol: Panthenol Available from Roche
*11Benzophenone-4: Uvnul MS-40 available from BASF
*12Octyl Methoxycinnamate: Parasol MCX available from Roche
*13Methylchloroisothiazolinone/Methylisothiazolinone: Kathon CG available from Rohm&Haas

Method of Preparation

The conditioning compositions of “Ex.1” to “Ex.7” as shown above can be prepared by any conventional method well known in the art. They are suitably made as follows:

Polymeric materials other than cationic crosslinked polymers, if included, are added to water at about 25-40° C. with agitation until they are dissolved. The mixture is heated to about 60-65° C. Temperature insensitive components such as parabens, if included, are added to the mixture with agitation until they are dissolved or dispersed. The mixture is cooled to about 50° C. Cationic crosslinked polymers are added to the mixture with agitation until they are dissolved. Then the mixture is cooled to below 40° C., and nonionic surfactants, and if included, remaining components such as silicones and perfumes are added with agitation. After it is homogenized, it is cooled to room temperature.

Examples 1 through 7 are conditioning compositions of the present invention which are particularly useful for hair conditioners for rinse-off use. These examples have many advantages. For example, the compositions of “Ex.1” through “Ex.7” have a suitable rheology for conditioning compositions, and provide conditioning benefits, especially softness and reduced tangling on wet hair when used for hair care products such as hair conditioning products. The compositions of “Ex.1” through “Ex.6” have transparent or translucent appearance, and the composition of “Ex.7” has opaque appearance. When used for hair care products, the composition of “Ex.1” through “Ex.7” can provide the above theological and conditioning benefits while not weighing down the hair. When used in a form of rinse-off products, the compositions of “Ex.1” through “Ex.7” can provide easy to rinse-off feel while providing the above theological and conditioning benefits.

All documents cited in the Detailed Description of the Invention are, are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A conditioning composition comprising by weight:
(a) from about 0.01% to about 5% of a cationic crosslinked thickening polymer;
(b) from about 0. 1% to about 10% of a surfactant selected from the group consisting of a nonionic surfactant, a cationic surfactant, and mixtures thereof; and
(c) an aqueous carrier;
and wherein the composition is substantially free of a water-insoluble high melting point oily compound.

2. The conditioning composition of claim 1 wherein the composition is transparent or translucent.

3. The conditioning composition of claim 1 wherein the composition is transparent.

4. The conditioning composition of claim 1 wherein the cationic crosslinked thickening polymer is a homopolymer of methyl quaternized dimethylaminoethyl methacrylate crosslinked by a crosslinking agent.

5. The conditioning composition of claim 1 wherein the cationic crosslinked thickening polymer has a crosslinked level of from about 50 ppm to about 1,000 ppm.

6. The conditioning composition of claim 1 wherein the surfactant is a nonionic surfactant.

7. The conditioning composition of claim 6 wherein the nonionic surfactant has an HLB value of from about 9 to about 18.

8. The conditioning composition of claim 7 wherein the nonionic surfactant is a polyoxyethylene alkyl ethers.

9. The conditioning composition of claim 1 further comprising from about 0.01% to about 5% of an additional thickening polymer.

10. The conditioning composition of claim 9 wherein the additional thickening polymer is selected from the group consisting of nonionic guar gum, nonionic cellulose polymer, and mixtures thereof.

11. The conditioning composition of claim 10 wherein the additional thickening polymer is a nonionic guar gum.

12. The conditioning composition of claim 1 further comprising from about 0.1% to about 10% of a silicone compound selected from the group consisting of a water-soluble silicone compound, silicone nanoemulsion, and mixtures thereof.

13. The conditioning composition of claim 1 further comprising from about 0.01% to about 10% of a cationic conditioning polymer.

14. The conditioning composition of claim 1 being substantially free of an anionic surfactant.

15. The conditioning composition of claim 6 being substantially free of a water-soluble cationic surfactant.

16. The conditioning composition of claim 1 being substantially free of a water-soluble salt.

17. The conditioning composition of claim 1 which is a hair conditioning composition.

18. The conditioning composition of claim 1 which is for rinse-off use.

19. A method of conditioning hair, the method comprising following steps:
(i) after shampooing hair, applying to the hair an effective amount of the conditioning composition of claim 1 for conditioning the hair; and
(ii) then rinsing the hair.
